(19) United States
(12) Patent Application Publication
(10) Pub. No.: US 2024/0164761 A1
(43) Pub. Date: May 23, 2024

(54) SURGICAL OR ENDOSCOPIC INSTRUMENT AND PRODUCTION THEREOF

(71) Applicant: KARL STORZ SE & Co. KG, Tuttlingen (DE)

(72) Inventors: Dominik BRAUNSCHWEIG, Tuttlingen (DE); Anton FOMIN, Tuttlingen (DE); Kamilla KÖNIG-URBAN, Tuttlingen (DE); Anna SCHWENDER, Tuttlingen (DE)

(21) Appl. No.: 18/282,957

(22) PCT Filed: Mar. 21, 2022
(86) PCT No.: PCT/EP2022/057330

§ 371 Date: Sep. 19, 2023

(30) Foreign Application Priority Data
Mar. 23, 2021 (DE) 10 2021 107 219.0

(51) Int. Cl. A61B 17/00; A61B 17/04; A61B 17/16; A61B 17/28

(52) U.S. Cl. CPC A61B 17/00234; A61B 17/0483; A61B 17/1613; A61B 17/1659; A61B 17/282; A61B 2017/00367; A61B 2017/00526; A61B 2017/00836; A61B 2017/2837

(57) ABSTRACT

A surgical or endoscopic instrument is provided with at least one longitudinal member (28, 30; 128; 228, 230; 328) having a longitudinal extension between a first end and a second end, and a support piece (50, 52; 150, 152; 250, 252; 350, 352; 410; 412; 414; 416), which has at least one structured contact section (60, 62; 160, 162; 260, 262; 360, 362; 420; 422; 424; 426) provided with toothing or corrugation, wherein the at least one structured contact section (60, 62; 160, 162; 260, 262; 360, 362; 420; 422; 424; 426) is integrated in the support piece (50, 52; 150, 152; 250, 252; 350, 352; 410; 412; 414; 416), wherein the support piece (50, 52; 150, 152; 250, 252; 350, 352; 410; 412; 414; 416) and the at least one structured contact section (60, 62; 160, 162; 260, 262; 360, 362; 420; 422; 424; 426) consist of a corrosion-resistant steel material, particularly a stainless steel, and wherein the at least one structured contact section (60, 62; 160, 162; 260, 262; 360, 362; 420; 422; 424; 426) is low-temperature diffusion hardened in a near-surface manner. Furthermore, the disclosure relates to a method for producing a surgical or endoscopic instrument.

The present disclosure relates to medical instruments, in particular surgical or endoscopic instruments, as well as a method for their production. In particular, the disclosure relates to instruments having increased hardness in the region of the contact surfaces with which the instrument interacts with tissue, organs or medical equipment.

A miniaturized instrument in the form of ocular forceps or pliers for eye surgery is known from WO 2020/004 667 A1. The instrument has a small-diameter shaft, on whose distal end there is constructed a miniaturized end effector having a fabric hinge. The shaft sits inside a thin sleeve that extends along the length of the small-diameter shaft between a proximal end of the shaft and the distal end, wherein there is provided on the proximal end an actuation handle that moves the sleeve proximally or distally relative to the shaft, wherein the sleeve on the distal end acts on the miniaturized end effector to open or close it.

A jawed instrument with structured gripping jaws is known from U.S. Pat. No. 6,077,280 A, said instrument being used for removing foreign objects from the human body. To grip and hold the foreign objects, the instrument has jaw members with so-called rat teeth, whose high and low points are matched to each other so that mutually opposed rat teeth of the jaw members can engage with each other in the closed state to secure foreign objects.

An instrument in the form of a needle holder is known from U.S. Pat. No. 6,322,578 B1, wherein the instrument has on its distal end two jaw members. It is proposed to provide separate inserts of hardened metal there so that the jaw members can grip surgical needles and the like. An ultrasound instrument is known from WO 2018/053 223 A1, which has an interlocking processing head on its distal end. The instrument is used for removing hard tissue or bone tissue. A surgical saw in the form of an oscillation tool is known from WO 2016/134 030 A2, wherein a sawblade is provided that consists of a stainless steel material.

Medical instruments, in particular surgical or endoscopic instruments, are sufficiently well-known. For example, these may be medical pliers, needle holders, forceps, files, rasps and similar instruments.

There are medical instruments that typically interact with soft tissue or other soft materials.

However, there are also medical instruments that encounter harder surfaces and parts. This pertains for example to so-called needle holders for guiding needles during surgical suturing. This also pertains, for example, to medical rasps and files (such as bone rasps and bone files), which are used for removing and processing bone and hard tissues. Such instruments therefore require contact surfaces and contact sections of corresponding hardness, to avoid becoming worn or even worn away by hard materials of that type.

This applies all the more when the instruments are provided with structuring in the region of their functional surfaces (contact sections), for example of an interlocking or corrugated form. Such a structure is used for the removal of material when rasping or filing, for example. With needle holders, pincers and similar instruments designed for gripping, structuring ensures a firm and secure grip.

In principle, medical instruments should be biocompatible and corrosion-resistant. Therefore, medical instruments of the aforementioned type are often produced out of low-corrosion or corrosion-free steel, particularly rustproof stainless steel. For one thing, this minimizes reactions with the body (of the patient). For another, such materials allow for (repeated) cleaning and sterilization.

Often, a certain elasticity and ductility (toughness) is desired because the medical instruments are often highly stressed and sometimes even deformed during use.

These conflicting demands for hard sections for gripping along with sufficient elasticity and toughness result in the fact that needle holders and similar instruments with high requirements in terms of surface hardness often have jaws/plates made of a different material with high hardness applied to a support (typically stainless steel). So-called hard metal plates or hard metal jaws are often installed which are attached to the support material in a materially-bonded manner (soldering, welding) for example.

In this way, the desired compromise of surface hardness and toughness can be achieved. However, hard metal parts are not entirely problem-free in terms of biocompatibility.

Furthermore, it has been shown that joining the hard metal parts to the support material (typically corrosion-resistant steel or stainless steel) can result in a visually adverse appearance that is not always accepted by the market.

With structured hard metal parts, for example ones with toothed or corrugated contact surfaces, it should be noted that while individual segments (teeth) may indeed be very hard, they can therefore also break under certain conditions.

With a stainless steel and hard metal composite, it must be kept in mind that during use and cleaning/sterilization, different heat expansion coefficients are present so that stresses may occur at the boundary between stainless steel and hard metal. This may even cause hard metal plates to become detached from their support material. For this reason, the joining process between hard metal and support material must be carried out with great care, which increases production costs.

Thus, the object of the present disclosure is to provide medical instruments, particularly surgical or endoscopic instruments, which have favorable properties in terms of biocompatibility and achievable surface hardness.

Preferably, a near-surface boundary zone with good surface hardness can be created while simultaneously ensuring the desired toughness/ductility in the core.

Such instruments should ideally be able to be produced with little effort. Furthermore, the instruments should have the most visually integrated design possible. The instruments should ideally be usable in a low-wear manner, and have a long service life and durability.

According to an initial aspect, the present disclosure relates to a surgical or endoscopic instrument designed as a jaw head instrument, having at least one longitudinal element extending between a first end and a second end, and with two jaw members that can be moved relative to each other, wherein each of the two jaw members forms a support piece that has at least one structured contact section, which is provided in particular with toothing or corrugation, wherein the structured contact sections of the two jaw members face each other at least in a closed state, wherein the structured contact sections are integrated into the respective support piece, wherein the respective support piece and the respective structured contact section consist of a corrosion-resistant steel material, specifically of a stainless steel, and wherein the structured contact sections are low-temperature diffusion hardened in a near-surface manner, specifically by a surface treatment using low-temperature diffusion hardening while forming a near-surface diffusion zone.

The object of the disclosure is achieved in this manner.

According to the disclosure, medical instruments designed in a sufficiently biocompatible manner can be provided in this way which also have outstanding surface hardness in the region of their structured contact sections.

In this way, the instruments can be subjected to high mechanical loads. At the same time, good tissue-compatibility is ensured. In addition, the instruments are suitable for intensive cleaning processes and in particular for sterilization.

Advantageously, the use of hard metal materials is avoided without having to forgo their favorable mechanical properties, particularly the hardness achievable in the region of the structured contact sections.

In an illustrative design, the structuring has, for example, one or two preferred directions, although this is not meant to be considered restrictive. The structuring may be designed in a similar manner to a knurl or cross-hatched knurl. However, the structuring may also have individual teeth arranged for example in rows with a preferred direction or with two or more preferred directions. Corrugation may have elongated notches, for example, arranged in one or two preferred directions. If there are two preferred directions, the notches may cross.

The instruments are designed in a corrosion-resistant and preferably rustproof manner, taking into consideration the conventional application conditions for medical instruments.

The support piece with the structured contact section can also be referred to as jaw or jaws, at least in illustrative designs.

The support piece and the structured contact section are integrally made of one and the same material. In other words, the structured contact section forms an integral component of the support piece. Thus, it is not a separate plate or the like connected to the support piece by means of a joining process.

Of course, the terms surgical and endoscopic do not have to be mutually exclusive, at least in the illustrative designs. Endoscopic surgical instruments are known. Generally, these are medical instruments that can be used close to the body or in the body. A medical instrument according to the disclosure has, for example, a distinct longitudinal extension between a proximal end and a distal end with one or more longitudinal members extending along the length.

Within the meaning of the present disclosure, the distal end is typically an end oriented away from the user and toward the patient. Within the meaning of the present disclosure, the proximal end is typically the end oriented toward the user and away from the patient. The distal end can also be referred to as the end near the patient. The proximal end can also be referred to as the end away from the patient. This shall not be understood as being restrictive, however.

The increase in hardness on the surface is made possible by forgoing a coating. This includes, for example, forgoing a diamond coating or a similar coating of hard cutting materials or other hardness-increasing substances.

The support piece and the structured contact section consist of the same material and are in particular produced in an integral manner. These are austenitic, rustproof stainless steels, at least in the illustrative designs.

Austenitic, rustproof stainless steels generally have high corrosion-resistance to water and various chemicals. However, the hardness and—associated with that—wear-resistance of the surfaces are limited. This is evident particularly when there is direct contact with hard objects. The hardening of such stainless steels using conventional heat-treatment processes requires considerable effort. At a minimum, one must expect unfavorable effects on corrosion resistance and possibly on visual appearance (coloring).

According to the present disclosure, the hardness-increasing surface treatment results from low-temperature diffusion hardening, which is designed particularly as a low-temperature carbon diffusion process. Comparable low-temperature diffusion processes for increasing hardness are designed as nitrogen diffusion processes and are comprised by the present disclosure.

In the illustrative designs, the surface treatment takes place according to so-called Kolsterising (a registered German trademark belonging to Bodycote plc, Macclesfield, Cheshire, Great Britain, at the time of filing this disclosure).

Low-temperature diffusion hardening processes include, for example, hardening of the boundary layer at less than 500° C. During hardening, considerable amounts of carbon can be diffused into the diffusion zone. The carbon is thereby released in interstitial spaces. Carbide formation is avoided. As a result, the diffused carbon results in increased compressive stresses in the near-surface region, so that a favorable surface hardness is created overall that is higher than the original state.

Additional advantages of the low-temperature diffusion hardening are favorable form stability and dimensional stability. Furthermore, specifically for austenitic stainless steels, unfavorable color changes can be avoided, which in conventional hardening processes would possibly have a negative effect on the visual appearance.

Instruments according to the disclosure contribute to the further replacement of hard metals in medicinal applications. At a minimum, the portion of hard metals and other foreign materials (for example, diamonds, cubic boron nitride and similar hard materials) can be further diminished.

Advantageously, corrosion-resistance is thereby retained. Biocompatibility is improved at least by the omission of potentially problematic materials.

According to another aspect, the present disclosure relates to a method for producing a surgical or endoscopic instrument according to at least one of the designs described herein, wherein the method comprises the following steps:
Providing at least one longitudinal member extending between a first end and a second end,
Providing a support piece made of a corrosion-resistant steel material, particularly stainless steel, wherein the support piece is designed as a section of the at least one longitudinal member or as a part coupled to the longitudinal member,
Creating at least one structured contact section on the support piece, particularly comprising the creation of toothing or corrugation, and
Performing a local surface treatment using low-temperature diffusion hardening while forming a near-surface diffusion zone for hardening the at least one structured contact section.

The object of the disclosure is also achieved in this manner.

The method for producing medical instruments according to the disclosure can be further developed along the lines of the designs and embodiments of the surgical or endoscopic instruments shown herein. Similarly, the instruments according to the disclosure can be further developed along the lines of the embodiments of the method according to the disclosure.

In particular, instruments according to the disclosure can be produced using the production methods according to the disclosure. The method according to the disclosure is suitable for producing and heat-treating medical instruments according to the disclosure.

According to an illustrative design of the instrument or the method, the support piece and the at least one structured contact section consist of a biocompatible steel material, particularly a biocompatible stainless steel.

Within the meaning of the present disclosure, the expression “biocompatibility” refers to the ability of the instrument to prevent adverse interactions with the body being treated during use. Stainless steels are generally considered to be sufficiently biocompatible for use in surgical or endoscopic instruments.

At least in the illustrative designs, the expression “biocompatibility” shall be understood within the meaning of the EU Medical Device Regulation ((EU) Regulation 2017/745). For example, by this standard, Standard ISO-10993 (version DIN EN ISO 10993-1:2010-04) explains the expression “biocompatibility” with respect to surgical or endoscopic use.

The instrument is designed, for example, as a needle holder. The instrument is designed, for example, as medical forceps. According to another alternative design of the instrument or method, the instrument is selected from the group consisting of the following: medical files and medical rasps.

In particular, these are medical instruments whose use brings them into interaction with or action upon sufficiently hard partners (materials, tissues and the like). This includes, for example, hard tissue such as bones, cartilage and the like. Furthermore, this may also involve medical products made of sufficiently hard materials, such as surgical needles, cannulas and similar.

Medical files or medical rasps are used, for example, for the targeted removal of bone, cartilage and other hard tissue.

Needle holders are used, for example, for gripping, holding and handling surgical needles.

These and similar medical instruments benefit from hardness-increasing measures in the region of the respective contact surfaces that come into contact with hard partners.

According to another illustrative design of the instrument or method, at least the support piece and the at least one structured contact section, preferably the entire instrument, are constructed to be free of hard metals.

At the same time, it is desirable for the structured contact section to have properties that are comparable to hard metals, particularly in regard to surface hardness. In other words, the instrument can forgo hard metal plates to form the structured contact section.

According to another illustrative design of the instrument or the method, the at least one structured contact section has an increased surface hardness.

Preferably, the hardening process occurs in such a way that primarily that region that is actually under a high load during use is subjected to a hardness-increasing treatment. Other regions are not required to be subjected to such a treatment.

According to another illustrative design of the instrument or method, the at least one structured contact section has a Vickers surface hardness of at least 750 HV 0.05, according to another illustrative design at least 850 HV 0.05, according to another illustrative design at least 950 HV 0.05, and according to another illustrative design at least 1050 HV 0.05. This applies to the state after carrying out the hardness-increasing surface treatment.

In this way, the instrument is suited for various applications in which an increased surface hardness is important, for example as a needle holder, medical file or rasp, and similar.

The Vickers hardness test under the HV 0.05 test condition is associated, for example, with a test force F at a level of approximately 0.4903 N (newtons). The hardness test HV 0.05 involves a microhardness test. The Vickers hardness test is suitable for surface-hardened work pieces. The Vickers hardness test is performed, for example, according to DIN EN ISO 6507-1:2018 to DIN EN ISO 6507-4:2018 standards.

Depending on the actual form, low-temperature diffusion hardening can generate maximum surface hardness in the range of 900 to 1300 HV 0.05, with the ductile properties of the core basically retained.

According to another illustrative design of the instrument or method, the depth of the diffusion zone in the structured contact section is 10 μm to 60 μm (micrometers), and according to another illustrative design 25 μm to 50 μm. In this way, one can ensure that the core retains its ductile properties. This reduces the instrument's susceptibility to breakage.

According to another illustrative design of the instrument or method, at least the support piece has a ductile core. Thus, in the core of the support piece, the base material has its original properties, and good toughness is provided. The risk of breakage is low.

According to another illustrative design of the instrument or method, the corrosion-resistant steel material is selected from a group consisting of the following: rustproof austenitic chrome-nickel steels having a low carbon content; rustproof austenitic chrome-nickel molybdenum stainless steels having a low carbon content; and corrosion-resistant heat-resistant iron-nickel-chrome steels.

An example of a rustproof austenitic chrome-nickel steel having a low carbon content is material number 1.4307. Examples of a rustproof austenitic chrome-nickel-molybdenum stainless steel having a low carbon content are material numbers 1.4435 and 1.4404. An example of a steel made from a corrosion-resistant, heat-resistant iron-nickel-chrome alloy is material number 1.4980.

According to another illustrative design of the instrument or method, the process temperature of the low-temperature diffusion process is selected to be below the recrystallization temperature of the steel material.

One advantage of the low-temperature diffusion process according to the disclosure is a very homogeneous, low-precipitation or precipitation-free diffusion zone, at least in illustrative designs. In this way, the favorable corrosion properties of the base materials are retained. The diffusion zone and the adjoining core zone are separated from each other in a sufficiently clear manner at least in illustrative designs. This can be shown by means of metallographic tests and comparable microstructure tests.

In conventional heat-treatment processes (for example, nitrocarburization), precipitates ensure that the separation between the diffusion and core zones is not so clearly marked. Thus, conventional heat-treatment processes result in decreased corrosion resistance.

According to another illustrative design of the instrument or method, the instrument is designed as a jaw-head instrument, particularly as a needle holder, wherein two jaw members are provided that are movable in relation to each other, wherein each of the two jaw members forms a support piece and is provided with a structured contact section, and wherein the structured contact section of the two jaw members face each other at least in the closed state.

Needle holders are specific surgical instruments designed in the form of pliers or scissors, for example, wherein two jaw members are provided which are meant to hold the needle with the appropriate holding force during surgical suturing. For this reason, the contact sections often have structuring. Furthermore, the contact sections should provide a certain surface hardness. Medical needles have a certain hardness. Medical needles and the like are meant to be held firmly and securely. Accordingly, high clamping forces act on the fine thin needles and the contact sections on the instrument.

Surgical suture needles often have special designs to enable using them in the most atraumatic (prevention of tissue injury) manner possible. This relates to their diameter (gauge), for one thing. Furthermore, various shapes are conceivable, for example a straight shape, slightly curved shape, highly curved shape and the like. One can also consider the fact that ideally there is no difference in diameter between the needle and the thread. Such special needles can for all practical purposes generally not be held and guided by hand. For this reason, various types of needle holders are known. The needle holders or their jaw members are specifically adapted to the needles used, at least in the illustrative designs.

Of course, applications other than suturing are conceivable, for example the setting of knots.

Of course, other jaw-head instruments are conceivable that have certain requirements in regard to surface hardness in the region of the jaws (with the structured contact sections). This relates, for example, to instruments whose purpose is to hold and guide other medical instruments/utensils.

Conventional needle holders typically have jaws made of hard metal materials, for example so-called sinter-metal jaws. These provide the desired hardness, but must be applied to the jaw members (support pieces). This is performed, for example, by means of welding or soldering. From a biocompatibility perspective, it is desirable to replace these hard metal parts.

The jaw members can move relative to each other at least between a closed state and an open state. In the closed state, a surgical needle or other surgical equipment, for example, can be firmly and securely held and guided.

According to another illustrative design of the instrument or the method, the longitudinal member is designed as a shaft that extends between a proximal end and a distal end, wherein a grip section is arranged at the proximal end, and wherein there are arranged in the distal end two jaw members, of which at least one jaw member can be moved, in particular pivoted, relative to the other jaw member.

An instrument designed in this manner is suitable, for example, for endoscopic or laparoscopic applications, in other words for use inside the body. Depending on the design, it is also suited for treatment of the body from outside the body.

According to another illustrative design of the instrument or the method, there extends through the shaft at least one actuating element for actuating the at least one movable jaw member. In this way, the instrument can be held and controlled by means of a handpiece with at least one handle on the proximal end, without the shaft necessarily moving when opening and closing the jaw members. Such a design is suited for laparoscopic and endoscopic instruments whose distal end is inserted into the body.

According to another illustrative design of the instrument or method, a ratchet is provided which secures the two jaw members in a closed state. In this way, it is ensured that, in the case of a needle holder for example, a fixed needle or the like is securely held.

According to another illustrative design of the instrument or method, the instrument is designed as a surgical file or rasp, wherein the longitudinal member is designed in a rod-like manner, wherein the at least one support piece is arranged as a blade at a first end or second end of the longitudinal member, and wherein the blade is provided on one side with a structured contact section.

Surgical files or rasps can be used, for example, to work on bones, cartilage or similar hard tissue. Therefore, greater surface hardness is advantageous in this case, too.

The transition from files to rasps is fluid. In terms of a definition, rasps generally refer to those instruments whose toothing is formed from individual teeth, which are positioned in a certain arrangement in an arbitrary or ordered manner. In terms of a definition, files generally refer to those instruments whose toothing is formed by rows of continuous (possibly crossed) lines. This shall not be understood as being restrictive. Various graduations between coarse and fine are conceivable in each case.

Surgical files or rasps that are subjected to high loads should have an adequate surface hardness. Likewise, high strength/toughness is desired in the core of the blade to prevent breakage and the like.

Therefore, conventional surgical files or rasps often also use hard metal jaws or hard metal blades that have the desired hardness properties, but that must be joined as a separate component to the tough rod-like longitudinal member.

According to another illustrative design of the instrument or method, the longitudinal member designed in a rod-like manner is respectively provided on its first end and on its second with a support piece designed as a blade, which has a structured contact section.

The structuring of the two contact sections placed on the ends facing away from each other can differ from each other. Thus, for example, a finely structured contact section and a coarsely structured contact section can be combined with each other.

Of course, the features mentioned above with reference to various embodiments and the ones still to be described below can be used not only in the respectively specified combination, but also in other combinations or singly, without departing from the scope of the present disclosure.

Additional features and advantages of the disclosure emerge from the description and explanation below of multiple illustrative embodiments with reference to the drawings.

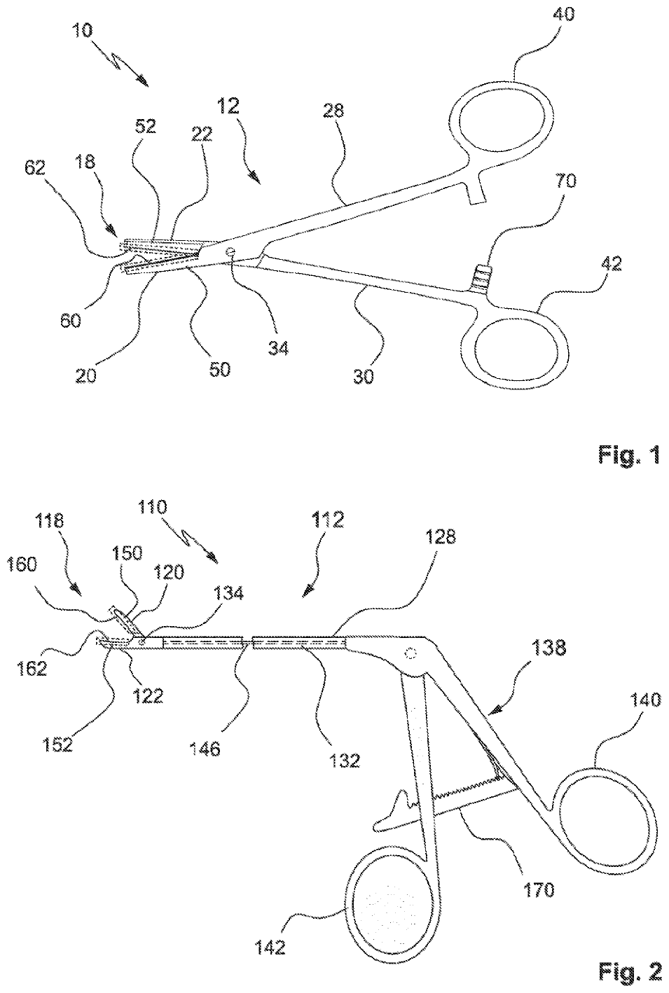

FIG. 1 depicts a longitudinal view of an embodiment of a medical instrument designed as a needle holder;

FIG. 2 depicts a broken longitudinal view of another embodiment of a medical instrument designed as a needle holder;

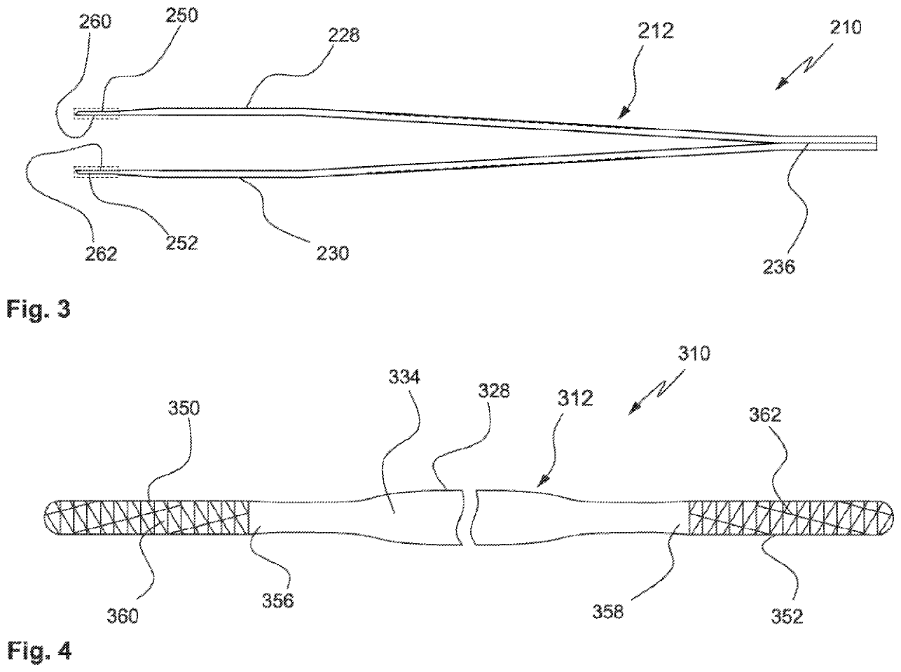

FIG. 3 depicts a longitudinal view of an embodiment of a medical instrument designed as forceps;

FIG. 4 depicts a broken longitudinal view of an embodiment of a medical instrument designed as a rasp or file;

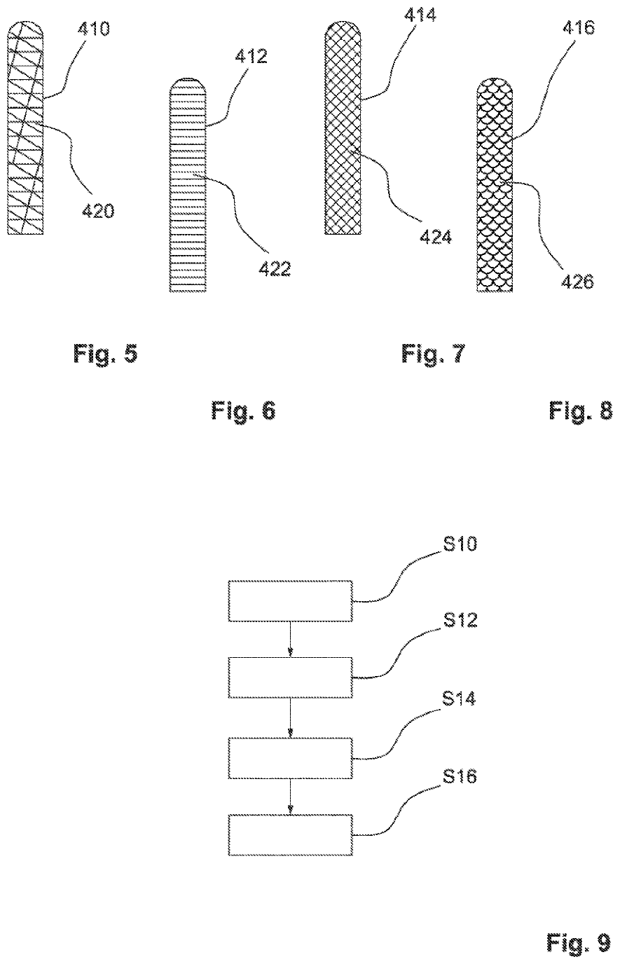

FIG. 5 depicts a schematic detail view of an embodiment of a structured contact section of a medical instrument;

FIG. 6 depicts a schematic detail view of another embodiment of a structured contact section of a medical instrument;

FIG. 7 depicts a schematic detail view of another embodiment of a structured contact section of a medical instrument;

FIG. 8 depicts a schematic detail view of another embodiment of a structured contact section of a medical instrument; and

FIG. 9 depicts a simplified block diagram to depict method steps of an embodiment of a method for producing a medical instrument.

FIG. 1 depicts a medical instrument labeled 10 as a whole, which is designed in the embodiment as a needle holder 12 with a scissor-like design. The instrument 10 comprises a jaw head 18 having a first jaw member 20 and a second jaw member 22. Between a proximal end away from the patient and a distal end near the patient, there extend two longitudinal members 28, 30, which in the embodiment are connected to each other in a hinged manner by means of a hinge 34.

The jaw head 18 with the jaw members 20, 22 is arranged on the distal end of the instrument 10. The longitudinal members 28, 30, each with a grip 40, 42, are coupled to the proximal end. In this way, a user can grip the instrument 10 with his hand and open and close the jaw head 18.

In the embodiment, the longitudinal member 28 has a support piece 50 on its distal end. The longitudinal member 30 has a support piece 52 on its distal end. The support pieces 50, 52 have contact sections 60, 62 facing each other, which are provided with structuring (not depicted in detail in FIG. 1). The structuring comprises, for illustrative purposes, toothing, corrugation or the like.

According to the disclosure, the support pieces 50, 52 with the contact sections 60, 62 are integrally designed out of one and the same base material. In the embodiment, the support pieces 50, 52 are integrally designed with the longitudinal members 28, 30 as components of the jaw members 20, 22 and formed out of one and the same base material. In particular, the base material is a low-corrosion or corrosion-free steel, for example a stainless steel. However, according to the disclosure, strength-increasing measures are provided to harden the surface of the support pieces 50, 52 at least in the region of the structured contact sections 60, 62.

In this way, the instrument 10 designed as a needle holder 12 can firmly and securely grip surgical needles and the like, without resulting in excessive wear of the contact sections 60, 62. To ensure the closed state of the jaw head 18, a ratchet 70 is provided in the embodiment which comprises a locking mechanism that is arranged on the proximal end of the longitudinal members 28, 30.

In a similar manner, FIG. 2 depicts a medical instrument labeled 110 as a whole, which is designed as a needle holder 112 in the embodiment. The needle holder 112 according to FIG. 2 is suited for endoscopic/laparoscopic applications. The instrument 110 comprises a jaw head 118. Furthermore, a longitudinal member 128 is provided which is designed as a shaft 132 in the embodiment. The longitudinal member 128 or shaft 132 has a marked longitudinal extension, in other words a large ratio between length and diameter. The image in FIG. 2 is broken along the length of the shaft 132. Consequently, the instrument 110 may have a greater length.

At the proximal end of the shaft 132, there is arranged a grip section 138 which comprises for example two arms, each forming a handle 140, 142. At least one of the handles 140, 142 is coupled via an actuating element 146 to the jaw head 118 to open and close the jaw head 118 (having two jaw members 120, 122) as needed. The actuating element 146 is, for example, a rod or a wire. The two jaw members 120, 122 can be pivoted relative to each other about a hinge 134, for example, to open and close the jaw head 118.

In the jaw head 118, the instrument has two support pieces 150, 152 with structured contact sections 160, 162 facing each other. The structuring comprises, for example, toothing, corrugation or the like. To secure the closed state, the embodiment has a ratchet 170 with a locking mechanism provided in the grip section 138.

The support pieces 150, 152 with the contact sections 160, 162 are each designed integrally with one of the jaw members 120, 122 and formed out of one and the same base material. To increase the hardness, a surface treatment is provided according to the disclosure by means of low-temperature diffusion hardening to form a near-surface diffusion zone, at least in the region of the structuring of the contact sections 160, 162.

FIG. 3 depicts another medical instrument 210 in the form of forceps 212. In the embodiment, the instrument 210 comprises a first longitudinal member 228 and a second longitudinal member 230, which can be referred to as arms or branches. The longitudinal members 228, 230 are connected to each other at the proximal end of the instrument 210 and form a base 236 there.

The longitudinal member 228 has on its distal end a support piece 250 having a structured contact section 260. The longitudinal member 230 has on its distal end a support piece 252 having a structured contact section 262. The contact sections 260, 262 each have structuring to simplify the gripping and holding of tissue, organs or medical equipment. The structuring is toothing or corrugation, for example. The structuring can be designed in the manner of a knurl or cross-knurl.

The longitudinal members 228, 230 are designed with sufficient elasticity or coupled to each other with sufficient elasticity so that the contact sections 260, 262 are pressed together by an external force on the instrument 210. According to the disclosure, the contact sections 260, 262 have a surface treated by means of low-temperature diffusion hardening to form a near-surface diffusion zone. The support pieces 250, 252 with the contact sections 260, 262 are integrally formed with the longitudinal members 228, 230, and specifically out of one and the same base material. Despite hardness-increasing measures in the region of the contact sections 260, 262, the longitudinal members 228, 230 are sufficiently elastic so that elastic deformation for opening and closing the instrument 210 is easily possible.

FIG. 4 depicts an instrument 310 that is designed as a medical rasp 312, particularly a so-called bone rasp. Alternatively, the instrument 310 may also be designed as a file or bone file.

The instrument 310 has a longitudinal member 328 designed as an elongated rod 324 (shown in FIG. 4 broken along its length) in the embodiment. The longitudinal member 328 extends between a first end and a second end. On each of the two ends, there is formed a blade 356, 358, which forms and supports a support piece 350, 352. The support piece 350 is provided with a structured contact section 360. The support piece 352 is provided with a structured contact section 362. In each case, the structuring comprises toothing with individual teeth or rows of teeth, corrugation or the like.

In the embodiment according to FIG. 4, the instrument 310 has on each of its two ends a structured contact section 360, 362. Accordingly, the instrument 310 is typically held and guided in a central region by a user. Of course, designs of bone rasps and similar medical files are conceivable in which only one of the two ends is provided with a structured contact section.

The structuring of the contact sections 360, 362 of the instrument 312 is used intentionally for removing material. Therefore, there is provided here, too, a surface treatment by means of low-temperature diffusion hardening according to the disclosure. In this way, the hardness in the near-surface region of the structured contact sections 360, 362 can be significantly increased. At the same time, the toughness in the core of the support piece 350, 352 or blades 356, 358 of the rod 334 of the longitudinal member 328 is retained.

In the design of the instrument 310 according to FIG. 4, in each case the end currently facing the patient (for example, to remove organic material such as bones or hard tissue) is the distal end. The corresponding end facing away from the patient is the proximal end. Utilization as the distal or proximal end can change accordingly.

The instruments according to FIGS. 1-4 have a significantly increased hardness in the region of their contact sections, at least at the surface of the respective structuring. This increase in hardness can be achieved by avoiding potentially problematic materials, such as hard metals and similar.

FIGS. 5-8 depict illustrative designs of structured contact sections for medical instruments. FIG. 5 depicts a support piece 410 having a structured contact section 420, wherein the structuring has two or more preferred directions. FIG. 6 depicts a support piece 412 having a structured contact section 422, wherein the structuring has a preferred direction and is designed in the manner of a knurl. FIG. 7 depicts a support piece 414 having a structured contact section 424, wherein the structuring has two preferred directions and is designed in the manner of a cross-knurl. FIG. 8 depicts a support piece 416 having a structured contact section 426, wherein the structuring has rows of individual teeth offset from each other. According to the disclosure, all the structured contact sections 420, 422, 424, 426 are provided with near-surface low-temperature diffusion hardening.

FIG. 9 depicts by means of a simplified block diagram an illustrative design of a method for producing a medical instrument, particularly a surgical or endoscopic instrument.

The method has a step S10, which comprises providing a longitudinal member for an instrument, for example providing an arm or support shaft for a jaw member instrument. Alternatively, step S10 comprises providing a rod-like longitudinal member for a medical file or rasp.

The method also has a step S12, which comprises providing a support piece out of a corrosion-resistant steel material, particularly a stainless steel. The support piece can be designed as an integral component of the longitudinal member. However, it is also conceivable to couple the support piece to the longitudinal member. On the basis of the selected material, the support piece has a sufficiently ductile core, yet has only a limited surface hardness.

The method also has a step S14 that comprises creating a structured contact section on the support piece, for example creating toothing or corrugation. In this way, the structuring can be created on the still-soft base material. In certain designs, step S14 precedes at least step S12 and possibly step S10. However, designs are also conceivable in which step S14 follows step S12 and possibly step S10.

The method has a step S16 which comprises a local surface treatment by means of low-temperature diffusion hardening while forming a near-surface diffusion zone. The surface treatment is executed particularly in the region of the structured contact section to harden it at least superficially. In this way, an overall favorable combination of hard surface and ductile core can be achieved in regard to the support pieces and the instrument as a whole.

### EXAMPLES

Support pieces in the form of jaw members made of selected low-corrosion materials having structured contact section were provided. The jaw members were subjected to a heat treatment according to the disclosure.

For the selected samples, the averaged values for the resulting surface hardness (HV 0.05) in the region of the structured contact sections were as follows:

TABLE 1

| Material | Surface-hardness treated [HV 0.05] | Diffusion depth (μm) |
|---|---|---|
| 1.4307 | 905 | 26 |
| 1.4404 | 1150 | 35 |
| 1.4435 | 1008 | 38 |
| 1.4980 | 1100 | 31 |

Thus, a significant increase of the surface hardness was achieved, specifically compared to the non-treated state. Preferably, a surface hardness can be achieved that is comparable to the hardness of hard metal plates in conventional medical instruments. The diffusion depths were visually measured by means of metallographic testing.

In addition, a hardness profile measurement was performed on samples that were heat-treated according to the disclosure to determine the diffusion depth in the hardened regions. For the mentioned materials, there were reproducible diffusion depths between 25 μm and 40 μm, in which the hardness of the boundary zone matches the hardness of the base materials. In illustrative designs, the objective is a diffusion hardness between 15 μm and 40 μm.

Therefore, the comparably small cross-sectional dimensions of the instruments according to the disclosure ensure that the core of the support pieces behaves in a sufficiently ductile manner. In certain designs, the diameter of the shaft or other longitudinal member of the instrument is less than 15 mm, in certain designs less than 12 mm, in certain designs less than 10 mm, in certain designs less than 8 mm, and in certain designs less than 6 mm. However, since only near-surface regions are intentionally hardened, the ductility of the core remains preserved even in such comparably thin workpieces.

All in all, one can achieve in this way a favorable combination of a hard surface in the region of the contact section and a tough, comparably elastic core. Thus, hard metals can be replaced for various medical instrument applications. The key point is that the surface hardness can be increased to such a degree that it permits the replacement of hard metal plates.

1. A surgical or endoscopic instrument comprising:
at least one longitudinal member with longitudinal extension between a first end and a second end, and
a support piece which has at least one structured contact section that is provided with toothing or corrugation, wherein the at least one structured contact section is integrated in the support piece, wherein the support piece and the at least one structured contact section include a corrosion-resistant steel material or a stainless steel, and wherein the at least one structured contact section is low-temperature diffusion hardened near the surface by a surface treatment using low-temperature diffusion hardening while forming a near-surface diffusion zone.

2. The instrument according to claim 1, wherein the support piece and the at least one structured contact section include a biocompatible metal material or a biocompatible stainless steel.

3. The instrument according to claim 1, wherein the instrument is selected from a group consisting of: needle holders, medical files, medical rasps and medical forceps.

4. The instrument according to claim 1, wherein at least the support piece and the at least one structured contact section are free of hard metals.

5. The instrument according to claim 1, wherein the at least one structured contact section has an increased surface hardness.

6. The instrument according to claim 1, wherein the at least one structured contact section has a Vickers surface hardness of: at least 750 HV 0.05, at least 850 HV 0.05, at least 950 HV 0.05, or of at least 1050 HV 0.05.

7. The instrument according to claim 1, wherein the depth of the diffusion zone in the structured contact section is 10 μm to 60 μm, or 25 μm to 50 μm.

8. The instrument according to claim 1, wherein at least the support piece has a ductile core.

9. The instrument according to claim 1, wherein the corrosion-resistant material is selected from the group consisting of: rustproof, austenitic chrome-nickel steels with low carbon content; rustproof, austenitic chrome-nickel-molybdenum stainless steels with low carbon content; and corrosion-resistant, heat-resistant iron-nickel-chrome alloys.

10. The instrument according to claim 1, wherein the process temperature of the low-temperature diffusion process is selected to be below the recrystallization temperature of the steel material.

11. The instrument according to claim 1, wherein the instrument is a jaw head instrument or a needle holder, wherein two jaw members are provided, which are movable relative to each other, wherein each of the two jaw members forms a support piece and is provided with a structured contact section, and whereby the structured contact sections of the two jaw members face each other at least in the closed state.

12. The instrument according to claim 11, wherein the longitudinal member is a shaft which extends between a proximal end and a distal end, wherein a grip section is arranged on the proximal end, and wherein two jaw members are arranged on the distal end, of which at least one jaw member is movable or pivotable, relative to the other jaw member.

13. The instrument according to claim 11, wherein an actuating element extends through the shaft and is configured to actuate the at least one movable jaw member.

14. The instrument according to claim 11, wherein a ratchet is provided which is operable to ensure a closed state of the two jaw members.

15. The instrument according to claim 1, wherein the instrument is a surgical file or rasp, wherein the longitudinal member is rod-like, wherein the at least one support piece is constructed on a blade on a first end or second end of the longitudinal member, and wherein the blade is provided at least on one side with a structured contact section.

16. The instrument according to claim 15, wherein the rod-like longitudinal member is provided on each of a first end and a second end with a blade having a support piece with a structured contact section.

17. A method for producing a surgical or endoscopic instrument, comprising:
providing at least one support piece having a longitudinal extension between a first end and a second end,
providing a support piece made of a corrosion-resistant steel material or a stainless steel, wherein the support piece is a section of the at least one longitudinal member or as a part coupled to the longitudinal member creating at least one structured contact section on the support piece, comprising toothing or corrugation, performing a local surface treatment by a low-temperature diffusion hardening while forming a near-surface diffusion zone for hardening the at least one structured contact section.
